(19) United States
(12) Patent Application Publication
(10) Pub. No.: US 2023/0148864 A1
(43) Pub. Date: May 18, 2023

(54) SYSTEM AND METHOD FOR MEASURING A FORCE EXERTED ON A BODY, AND FOR CHARACTERIZING THE RIGIDITY OF A BODY USING SUCH A DEVICE

(71) Applicant: Anatoscope, Montpellier (FR)

(72) Inventor: François Faure, Crolles (FR)

(21) Appl. No.: 17/759,299

(22) PCT Filed: Jan. 21, 2021
(86) PCT No.: PCT/FR2021/050108

§ 371 Date: Jan. 30, 2023

(30) Foreign Application Priority Data
Jan. 23, 2020 (FR) 2000652

(51) Int. Cl. A61B 5/00

(52) U.S. Cl. CPC A61B 5/0053; A61B 5/0033

(57) ABSTRACT

A device for measuring a force exerted on a body comprises a flexible rod having a proximal end and a distal end intended to come into contact with the body and to apply a force tending to deform the body near a point of contact. The measuring device also comprises an image capturing device, which is rigidly attached to the proximal end and oriented toward the distal end of the flexible rod, for providing at least one depth map of the body near the distal end of the flexible rod. The device may be used to perform a method of measuring the force exerted on the body and a method of characterizing the rigidity of a body

### CROSS-REFERENCE TO RELATED APPLICATIONS

This application is a national phase entry under 35 U.S.C. § 371 of International Patent Application PCT/FR2021/050108, filed Jan. 21, 2021, designating the U.S. of America and Published as International Patent Publication WO 2021/148755 A1 on Jul. 29, 2021, which claims the benefit under Article 8 of the Patent Cooperation Treaty to French Patent Application Serial No. FR2000652, filed Jan. 23, 2020.

### TECHNICAL FIELD

The present disclosure relates to a system and a method for measuring a force exerted on a body. This system and this method find applications in the field of characterizing and modeling the rigidity of a body.

### BACKGROUND

There are a multitude of situations in which it is useful to have a characterization of the rigidity of a body.

In the medical field, it is common to examine a patient by palpation, that is, by direct physical contact. The distribution of the rigidity in the part of the examined body allows the practitioner to identify regions of the patient’s body that are likely to be abnormal, for example, comprising tumors.

Measurement of the rigidity of a part of a human body can find other applications, for example, to design an object intended to be worn by a user (for example, a prosthesis, an orthosis, or even a garment) that is not only fitted to the user’s morphology, but that also takes into account the deformation of the user’s body when the prosthesis, the orthosis or the garment is placed on the user’s body and exerts pressure on this body.

Rigidity measurement is also useful for the complete modeling of a deformable body (a human body or a body of any other nature), the model then representing the deformation behavior of this body when it is subjected to outside forces. The model can be useful to allow faithful replication of this object.

As recalled in the document “Capture and modeling of non linear heterogeneous soft tissue,” by B. Bikel et al. ACM Transactions on Graphics 28(3):89, July 2009, the work of characterizing and/or modeling a deformable body can implement a system for measuring the deformation of the body when the latter is subjected to a force. More specifically, the measurement system proposed by this document comprises a contact probe for exerting a force on the body from time to time. The probe comprises a force sensor for measuring the force applied by the distal part of a rigid rod provided with an application tip. The system also comprises 3 imaging devices for recording the deformation of the surface of the body from three different angles, when the probe exerts a force thereon. The activation of a trigger by the operator of the contact probe makes it possible to simultaneously measure the force applied and to take 3 shots. Markers are painted on the body and on the rod of the probe in order to identify, by image processing, the deformation of the surface and the position/orientation of the probe.

A similar measurement system is described in document US9623608. This system comprises a robot arm provided with a force sensor, the arm being configured to apply a force to the body or to the object being tested. The system also comprises a plurality of imaging devices, arranged in a semi-circle above the arm of the robot, to track the deformation of the object during the measurement sequence. The object can carry means for assisting the processing of images produced by the measurement system.

Document US 2011/0319791 discloses a system suitable for measuring the mechanical properties of a deformable material.

The document “The human touch: Measuring contact with real human soft tissues” by Pai et al, ACM Transactions on Graphics. 37. 1-12. (2018) proposes a portable contact probe comprising a head bearing three RGB cameras and a rigid rod, connected to the head, associated with a force and torque sensor. The distal end of the rod is intended to come into contact with a patient’s body so as to apply force and deform it. A tracking system captures images via three cameras placed in the room, to enable the probe to be located and tracked in a coordinate system linked to the world. These images are digitally processed to establish an optical flow (i.e., the apparent movement of the body under the effect of the application of force) that makes it possible to analyze the displacement of the surface of the patient’s skin.

The document by Faragasso et al. “Real-Time Vision-Based Stiffness Mapping” (2018), Sensors, 18, 1347 describes a device for measuring the deformation of a body or an object. The device comprises a plurality of punches respectively mounted on springs and connected to a base. When the end of the punches is applied against the object or the body, the springs contract based on the force applied, their stiffnesses and the rigidity of the body of the object at the respective points of contact. The measuring device is provided with an image capturing device making it possible to capture an image of the springs in order to identify their contractions and thus to determine the deformation of the body at each point of contact with the punches. Solving a system of nonlinear equations makes it possible to determine the stiffness of the object, assuming that this stiffness is the same under each punch.

These prior art devices are particularly complex to implement. Some of them require identifying the position and orientation of the contact probe in the images provided by the cameras to reconstruct all the components of the force exerted on the body. The device described by the Faragasso document requires applying the force in the direction of contraction of the springs; this solution does not take into account the existence of tangential components of the force applied. Furthermore, the application of the force is carried out over a relatively large area of the body, which does not allow the point of application of this force to be properly located and limits the resolution of the rigidity analysis.

An object of the present disclosure is to remedy, at least in part, the limitations of this state of the art. It aims, in particular, to provide a system for measuring a force exerted on a body that is simple to implement. This system can be used to characterize the rigidity of a body with satisfactory resolution.

### BRIEF SUMMARY

With a view to achieving one of these aims, the object of the present disclosure proposes a measuring system intended to measure a force exerted on a body; the measuring system comprises a measuring device and calculation means;
the measuring device comprises:
   a flexible rod having a proximal end and a distal end intended to come into contact with the body and to apply a force tending to deform it near a point of contact, the flexible rod being configured to bend as soon as it applies a force by its distal end to the body;
   an image capturing device secured to the proximal end and oriented toward the distal end of the flexible rod to provide at least one depth map of the body near the distal end of the flexible rod, and the distal end;
the calculation means are configured, from the depth map of the distal end of the rod, to determine the force exerted by the flexible rod by its distal end on the body.

According to one embodiment, the flexible rod is configured to apply a force between 0.1 N and 10 N.

According to one embodiment, the measuring device comprises a switch to actuate the image capturing device.

According to one embodiment, the system further comprises a gripping handle secured to the image capturing device and to the flexible rod.

According to one embodiment, the switch is in the form of a trigger.

According to one embodiment, the calculation means comprise a transmission unit and a remote calculation device, the transmission unit being configured to exchange data with a remote calculation device.

According to one embodiment, the calculation means comprise a calculation unit configured to process the depth map.

According to one embodiment, the image capturing device also provides a digital image of the body near the distal end of the flexible rod.

According to one embodiment, the distal end comprises a tip having a color so as to be easily identified in the image provided by the image capturing device.

According to one embodiment, the distal end comprises a tip having a shape chosen to be easily identified on the map provided by the image capturing device.

The present disclosure also relates to a method for measuring a force exerted on a body, the method comprising:
a step of applying the measurement system according to the present disclosure so that the distal end of the rod contacts the body at a point of contact and applies a force tending to deform the body near the point of contact;
a step of acquiring, at a measurement instant, at least one depth map during the application step;
a step of processing the depth map to establish the force exerted on the body.

According to one embodiment, the processing step comprises a sub-step of locating the distal end of the rod in the depth map to provide coordinates of this end in a coordinate system associated with the measuring device.

According to one embodiment, in which the method comprises a preliminary step of calibrating the measuring device.

The present disclosure also relates to a method for characterizing the rigidity of a body comprising acquiring, during a measuring method according to the present disclosure, a plurality of depth maps at successive measurement instants.

According to one embodiment, the characterization method comprises a step of readjusting the plurality of depth maps.

According to one embodiment, the body comprises readjustment markers making it possible to readjust the plurality of deformation maps.

### BRIEF DESCRIPTION OF THE DRAWINGS

Other features and advantages of the present disclosure will become apparent from the following detailed description of example embodiments of the present disclosure, which is provided with reference to the accompanying figures, in which:

FIG. 1 shows a system for measuring the force exerted on a body according to the present disclosure.

FIGS. 2A-2C schematically and respectively illustrate the behavior of the measuring device before contact, in simple contact and in forced contact with a body being tested;

FIGS. 3A-3C show an example of the processing implemented during a method according to the present disclosure; and

FIGS. 4A to 4C show an example of the processing implemented during a method for characterizing the rigidity of a body according to one aspect of the present disclosure.

### DETAILED DESCRIPTION

### System for Measuring the Force Exerted on a Body

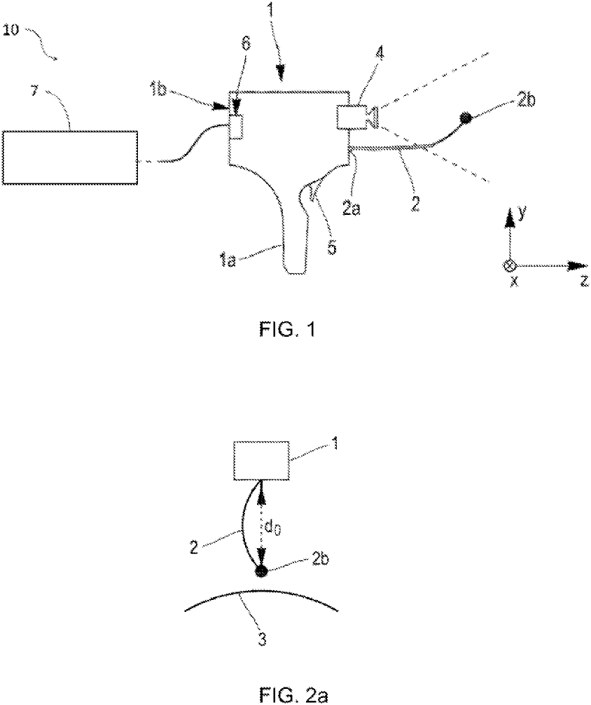

In the example shown in FIG. 1, a measuring system 10 that comprises a measuring device 1 and calculation means 7. The measuring device 1 according to the present disclosure is formed by a head 1b and a gripping handle 1a. The head 1b bears a flexible rod 2 having a proximal end to which the head 1b is connected. A distal end 2b of the flexible rod 2 is in turn intended to come into contact with a body being tested. More precisely, the measuring device 1 is manipulated by an operator to approach the distal end 2b of the body, so as to come into contact with it. In this way, a force is transmitted and applied to the body, tending to deform it near the point of contact.

For safety reasons, as well as to avoid damaging the body at the point of contact, it is possible to provide the distal end 2b with a protective tip. This may have a contact surface, that is to say, a surface intended to come into contact with the body, that is flat or rounded so as to ensure this protective function, but without being too large to preserve the pointed nature of the contact that forms between the distal end 2b of the flexible rod 2 and the body being tested.

By way of example, the protective tip may correspond to a plastic sphere embedded on the distal end 2b of the flexible rod, and having a diameter of the order of a few millimeters, for example, comprised between 2 mm and 5 mm. As will be detailed later in this presentation, the shape of the tip and/or its color can also be chosen to be easily identified on a depth map or an image that is acquired during use of the measuring device 1.

As has already been mentioned, the rod 2 makes it possible to transmit a force applied to the measuring device 1 and to apply this force to the body. This rod 2 is flexible, that is to say, the reaction force that is applied to the distal end 2b of the rod 2 when the latter is in forced contact with the body tends to deform it, for example, by bending. To a certain extent, the flexibility of the rod 2 makes it possible to make the displacement of the distal end 2b of the rod independent of the displacement of the head 1b of the measuring device 1 on which it is fixed. The component material of the flexible rod 2 and its shape are chosen so that, in the range of forces that it is desired to apply to the body, the rod 2 can deform elastically. When the body is a human body, this force can be between 0.1 N and 10 N.

It is thus possible to provide for the rod 2 to be formed of a metal or of a plastic. It may also have a bend or be curved (as shown in FIG. 1) to promote its elastic bending when the measuring device 1 is applied to the body being tested. The curved shape of the rod 2 can also have the advantage of placing the distal end 2b, when the rod 2 is not deformed, in the center (or close to the center) of the field of vision of an image capturing device 4 of the measuring device 1, as will be detailed later in this description.

Continuing the description of FIG. 1, the measuring device 1 also comprises an image capturing device 4, here a camera integrated into the head 1b of the device 1. The head 1b makes it possible to fix the relative positions of the image capturing device 4 and of the proximal end 2a of the rod 2 so as to secure them to one another. The head 1b does not, however, form an essential element of the measuring device 1 and one could envisage other configurations of this device 1 in which the image capturing device 4 is secured to the proximal end 2a of the rod 2 without requiring the presence of a head. The proximal end 2a of the rod 2 could, for example, be attached directly to the image capturing device 4 or to a housing of this device 4.

Regardless of how these two elements are secured to each other, the image capturing device 4 is oriented toward the distal end 2b of the flexible rod 2. In other words, the orientation of the image capturing device 4 is such that the distal end 2b of the flexible rod 2 is in its field of vision, that is to say, in the area of the space that the sensor(s) of the image capturing device 4 perceives. As mentioned above, the measuring device 1 can be configured so that, at rest, the distal end 2b is placed in the center, or near the center, of this field of vision.

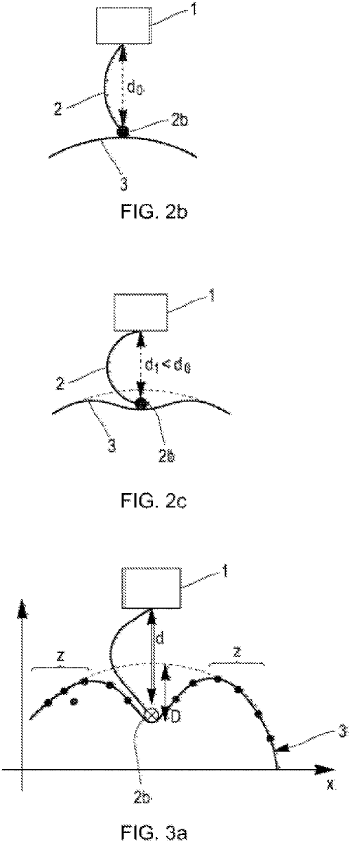

When the measuring device 1 applies a force to the body being tested (as will be presented in connection with the description of FIG. 2), the orientation of the image capturing device 4 toward the distal end 2b of the rod 2 also causes a portion of the body located near the point of contact to enter the field of vision of the image capturing device 4. Since this area around the point of contact is likely to be deformed by the application of the force, the arrangement of the image capturing device 4 also makes it possible to capture a deformation profile and an image of the deformation of the body being tested.

According to the present disclosure, the image capturing device 4 is provided with a sensor capable of providing a depth map. The sensor performs the measurement in a plane, called “image plane,” which is provided with a coordinate system (x,y) referred to as “coordinate system of the image capturing device” in the remainder of this description. This coordinate system can be increased by a depth direction z, perpendicular to the image plane, in order to form a complete coordinate system (x, y, z). Since the image capturing device 4 is secured, without any degree of freedom, to the head 1b of the measuring device 1, the coordinate system of the image capturing device also forms a coordinate system linked to the measuring device 1.

The depth map provided by the image capturing device 4 can take the form of a scatter chart whose coordinates are established in the complete coordinate system (x, y, z). The map may alternatively be in the form of an image comprising elements of the image plane of the sensor respectively associated with points of the imaged scene, each element of the image plane also being associated with distance information separating this plane of the image capturing device 4 from the point of the scene in question.

Advantageously, but without this forming an essential feature of the present disclosure, the image capturing device 4 can also provide a digital image of its field of vision. To this end, the image capturing device 4 may have an optic, or any other means, making it possible to take an image of the deformed surface of the body being tested as well as of the distal end 2b of the rod 2. The focal distance of the optic can be chosen according to the extent of the desired field of vision and therefore the extent of the imaged surface of the body being tested. A depth of field can advantageously be chosen that is sufficient for it to comprise the distal end 2b of the flexible rod 2 for the entire range of forces applicable on the body, and therefore for the entire bending range of the rod 2.

For the sake of clarity, it is recalled that a digital image is defined by image elements, referred to as “pixels,” that carry at least one digital value of light intensity of gray levels or of color. Pixels are usually arranged in the form of a matrix, in rows and columns. Such a digital image can, however, be recorded in the image capturing device 4, and be transmitted in another form, for example, a compressed form, but in all cases the image can be manipulated to represent an image composed of pixels arranged in a matrix.

When the image capturing device 4 combines both distance information and color or light intensity information, this device can be formed, for example, by an RGB-D camera, a description of which is found in documents US20070216894 and US7433024. In addition to the traditional image sensor, for example, allowing determination for each pixel of a radiation intensity value in the red, green and blue range, an RGB-D camera also projects infrared radiation whose reflection is captured by an infrared image sensor. The measured radiation is processed to determine the distance separating the sensor from any objects placed in the field of vision, according to, for example, the technology described in the aforementioned documents. The infrared sensor and the image sensor are calibrated together, so that it is possible to associate depth information with intensity information for each pixel.

It should be noted that the data from the image capturing device 4, according to the present disclosure, are provided in any format, which is not limited to that of a digital image whose pixels are augmented with depth information. This data can also be presented, for example, as already mentioned, in a scatter chart format. In such a format, each point of the scatter chart is represented by its coordinates in the complete coordinate system (x, y, z), possibly enhanced by color information.

Whatever the format in which the data established by the image capturing device 4 are conditioned, they will be referred to by the terms “images” for the data relating to the intensity of the visible radiation perceived on the image plane of the image sensor, and by “depth map” for the data relating to the distance separating the objects of the scene from the image capturing device 4. And these data are identified in the same coordinate system associated with the image capturing device 4, and therefore with the measuring device 1.

The image capturing device 4 can make it possible to take a succession of images and depth maps, for example, every 20 ms, to constitute an animated sequence. When the measuring device 1 is used to actually analyze the deformation of a body, the image capturing device 4 provides data that can be represented in the form of an animated sequence of the deformation of this body, and comprises both images and successive depth maps of the deformation.

To initiate the capture of an image and/or a map, or of a sequence of such images and such maps, the measuring device 1 can be provided with a switch 5 that can be actuated by the operator. The operator can manually actuate, for example, the switch and trigger the capture(s), while he moves the measuring device 1 toward the body being tested and applies a force to this body, tending to deform it. At the end of this step, one then has an animated sequence of images and a depth map imaging the deformation of the body. These images of course comprise the distal end 2b of the flexible rod 2, which is placed at all times in the field of vision of the image capturing device 4.

For practical reasons, provision may be made for the switch 5 to take the form of a trigger placed on the gripping handle 1a of the measuring device 1, as shown in FIG. 1. This allows the operator to use the measuring device 1 with one hand and to actuate the switch 5 while manipulating the device 1 to apply it to the body being tested.

The data produced by the image capturing device 4, digital images and depth maps or any other data produced from this information, can be communicated to the calculation means. The calculation means can in this respect comprise a remote calculation device to be exploited there and a transmission unit. For this purpose, the measuring device 1 can comprise a transmission unit 6 connected to the image capturing device 4. This transmission unit 6 can take the form of a simple connector making it possible to plug in a connecting cable between the measuring device 1 and the calculation device. It may alternatively be a more complex electronic circuit making it possible to condition the data before transmitting them, for example, using a wireless link, to the calculation device. The transmission unit can also make it possible to configure and/or operate, from the remote calculation device, the image capturing device 4 and more generally the measuring device 1.

The calculation means can also comprise a calculation unit secured to the measuring device 1. The calculation unit can comprise, for example, a microcontroller, a graphics chip, an FPGA, specifically configured to carry out processing operations on the data supplied by the image capturing device 4, before these processed data are transmitted to the remote calculation devices.

These processing operations can correspond to simple formatting of the images and of the depth maps provided by the image capturing device 4, for example, to provide them in the form of a scatter chart of colored points as mentioned above, or to reduce the resolution thereof, or else to extract richer information therefrom, such as, for example, the force exerted by the flexible rod 2 on the body being tested that is the subject of the measuring method, the description of which follows.

FIGS. 2A-2C schematically show the behavior of the flexible rod 2 before the distal end 2b comes into contact with a body 3 being tested, at the moment of contact of this distal end 2b with the body 3, without, however, applying forces on this body, and when the measuring device 1 is brought close enough to the body 3 for a force to be applied thereto.

In FIGS. 2A and 2B, no force is therefore applied to the body 3 and the latter therefore has its “natural” shape, without deformation. This is also the case for the flexible rod 2. The distal end 2b is therefore separated from the measuring device 1 by a distance at rest d0. This distance can be, for example, that separating the distal end 2b from the proximal end 2a of the flexible rod 2, or from any other fixed point of the measuring device 1.

In FIG. 2C, the measuring device 1 is brought close enough to the body 3 for the rod 2 to apply a force to this body 3 at the point of contact. The body 3 is deformed at and near the point of contact. Since the rod 2 is flexible, the latter is also deformed in bending, so that the distance d1 separating its distal end 2b from its proximal end 2a is reduced, compared to the distance at rest d0 of FIGS. 2A and 2B.

It is noted that, knowing the distance separating the distal end 2b from the measuring device 1, it is possible to unequivocally determine the force applied by the rod 2 (and therefore by the measuring device 1) on this body 3.

In the illustration of FIGS. 2A-2C, the measuring device 1 is brought closer to the body 3 to apply a force, via the rod 2, that has no tangential component. More generally, the measuring device 1 can be brought closer to the body 3 in various directions in order to impart a force having a tangential component to the body 3. In this case, the distal end 2b of the flexible rod 2 moves not only along a depth direction z in the complete coordinate system associated with the device, but also along directions perpendicular to this depth direction z. In other words, the coordinates of the distal end 2b of the rod 2 are likely to vary according to the three components (x, y, z) in a coordinate system linked to the measuring device 1 when the latter is moved to apply a force on the body using the flexible rod 2.

In this general approach, it is noted that it is possible to determine all the components of the force applied to the body 3 by the measuring device 1 from the position of the distal end 2b of the rod 2 in the coordinate system linked to the measuring device 1.

In certain simple cases, the relationship linking the position of the distal end 2b to this force can be formulated explicitly, based on, for example, the dimension of the rod 2 and/or stiffness coefficients. More generally, though, this relationship can be established by a prior calibration step during which the measuring device 1 is applied to a force sensor pre-calibrated according to 3 components (known as a “tri-axis” sensor).

For example, the sensor can be carried by a rigid plate that can be moved in a controlled manner in 3 orthogonal directions. The measuring device 1 is fixedly arranged opposite the plate and in line with the force sensor so that the distal end 2b of the flexible rod 2 comes into contact with this sensor. A battery of measurements is then taken by moving the rigid plate in the 3 possible directions so as to collect:
the components fx, fy, fz of the force applied by the flexible rod 2 to the sensor, and such that these components are provided by the sensor;
the coordinates (x, y, z) of the distal end 2b of the rod 2 in the coordinate system linked to the image capturing device 4. These coordinates can be recorded manually from the depth map provided by this device 4 or by digital processing of this map, as will be explained in another part of this description.

In this way, it is possible to collect pairs of measurements {(x, y, z); (fx, fy, fz)} connecting the position (x, y, z) of the distal end 2b of the rod 2 to the components fx, fy, fz of the force applied by this rod 2. These pairs of measurements can be used to determine, by interpolation, the components of the force applied by the measuring device 1 to the body for any position of the distal end 2b in a coordinate system linked to the measuring device 1 (such as that associated with the image capturing device 4).

In other words, in a measuring device 1 according to the present disclosure, the position of the distal end 2b of the rod 2 with respect to a reference point of the measuring device 1 can be connected unequivocally to the components of the force applied by this device 1 to a body 3.

This characteristic of the measuring device 1 is exploited in order to provide a method for measuring the force exerted on a body 3 that is particularly simple to implement, which is the subject of the following section of this description.

### Method for Measuring the Force Exerted on a Body

The measuring method comprises a first step of applying a measuring device 1, as described previously, to a body 3 being tested. As already mentioned, during this step, the measuring device 1 is brought closer to the body 3 so as to bring the distal end 2b of the flexible rod 2 into forced contact on this body 3. A force is therefore exerted on the body 3, this force tending to deform the body 3 at and near the point of contact. The flexible rod 2 is also deformed. This step can be implemented by an operator or, alternatively, by an automated system, such as a robot arm whose end would be equipped with the measuring device 1.

Once the forced contact of the measuring device 1 on the body 3 has been established, the device 1 can be moved away from the body 3 and the method can be interrupted, or repeated at an identical or different point of contact.

The measuring method also comprises an acquisition step during which at least one depth map is acquired, at a measurement instant, using the image capturing device 4. As already mentioned, a plurality of maps will preferably be acquired at successive measurement instants, so as to collect an animated sequence of the deformation of the body 3 being tested caused by the forces exerted by the measuring device 1 over time.

The acquisition step can be triggered and interrupted by the switch 5 or by the trigger of the measuring device 1 operated by the operator during the application step. Alternatively, the acquisition can be triggered by the remote calculation device to which the measuring device 1 can be connected, in particular, when the latter forms part of an automated measuring system.

Regardless of how the acquisition step is triggered, it will be ensured that it is temporally included in or overlapping the step of applying the measuring device 1 so that it is possible to proceed to the acquisition of at least one depth map of the body while the flexible rod is in forced contact with this body.

When the image capturing device 4 allows it, this acquisition step also carries out the acquisition of digital images of the body 3 simultaneously with the acquisition of the depth maps.

The data acquired during this step can be stored in a buffer memory of the image capturing device 4 or of the measuring device 1. They can be processed by the calculation unit and/or transmitted to the remote calculation device with a view to being processed there via the transmission unit 6 to which the image capturing device 4 is directly or indirectly connected.

Whether these data are processed locally or by the remote calculation device, a measuring method according to the present disclosure comprises a step of processing these data (at least one depth map and, optionally, at least one digital image) aimed at establishing the force exerted by the measuring device 1 on the body 3, at the measurement instant.

To this end, the processing step comprises a first sub-step aimed at locating, via a digital processing technique, the distal end 2b of the flexible rod 2 in the depth map or, when the latter is available, in the digital image. This sub-step can be facilitated by the presence of a protective tip having a specific shape, making it possible to clearly distinguish its outline in the depth map. This location sub-step can comprise conventional digital processing in the field of computer vision and can be implemented, for example, by a neural network. At the end of execution, this sub-step provides the coordinates, in the coordinate system (x, y, z) associated with the image capturing device 4, of the distal end 2b of the rod 2.

When this processing is done on the digital image, the tip can be chosen to also have a specific color, thus facilitating its location in the image. The location of the tip can very simply be expressed in the form of the column and row ranks of a pixel of the image. The rows of the pixel and the depth map are then used to determine the coordinates, in the coordinate system (x, y, z) associated with the image capturing device 4, of the distal end 2b of the rod 2.

If the depth map has a lower resolution than that of the image, this sub-step may comprise or correspond to the search for the point of the depth map closest to the coordinates identified in the previous sub-step, in order to take consistent information from the depth map.

In all cases, and regardless of the processing implemented during this sub-step, the outcome of this sub-step is provided with the coordinates, in the coordinate system (x, y, z) associated with the image capturing device 4, of the distal end 2b of the rod 2, at the measurement instant. When several successive measurements are carried out, a list of these coordinates is available.

In a second sub-step of the processing step, the position of the distal end 2b of the rod 2 at the measurement instant is converted into a force. To perform this conversion, the function or the calibration table of the flexible rod 2 is used, associating the position of the distal end 2b with an applied force. As seen previously, this function or this table may have been developed in a calibration step, prior to the steps making up the measuring method itself.

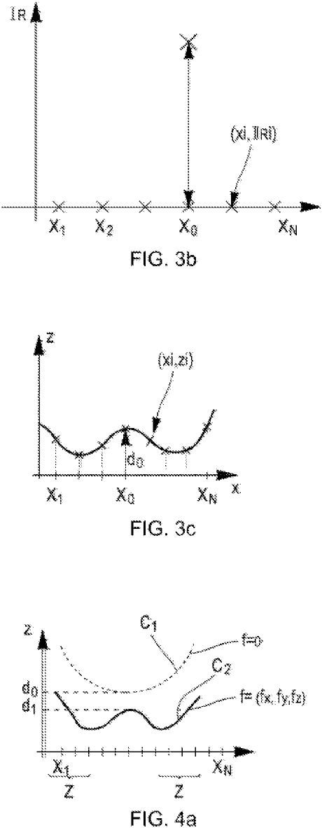

FIGS. 3A to 3C thus illustrate an example of implementation of the processing operations that have just been described. In this example, the image capturing device 4 corresponds to an RGB-D camera and the color information provided by the digital images is used to identify the tip of the flexible rod 2. FIG. 3A shows the measuring device 1 in forced contact with a body 3 at the distal end 2b of the flexible rod 2.

To simplify the illustration, the body 3 being tested here has a single dimension, and an image of this body is therefore composed of pixels x1 to xn arranged in a line along an axis X that forms the coordinate system of the image capturing device 4. Each pixel xi is, in this example, associated with a measurement of red color intensity, provided by an image sensor of the image capturing device 4, and with a depth measurement z, provided by, for example, a device with an infrared image sensor.

In the illustrated example, it is considered that the body 3 has an exposed surface essentially of blue color, which contrasts well with the red tip of the flexible rod 2 and which facilitates the identification of this tip in the image.

The illustration of FIG. 3C shows the depth map, here the distance zi associated with each pixel xi, and the illustration of FIG. 3B shows the red intensity image IR, that is to say, the intensity of red color I_{Ri} associated with each pixel xi.

The first location sub-step is facilitated by the choice of the very distinct color of the distal end 2b of the rod 2 in comparison with that of the body 3, so that in the image composed of pixels associated with an intensity of red color, it is easy to locate the pixel x0 whose intensity in the red is well marked with respect to the other pixels that are in turn linked with the body 3 being tested, here blue in color. In practice, algorithms from the field of computer vision can be implemented to identify the distal end 2b of the rod 2 in the image, and to choose a pixel representative of the position of this end in the image.

Knowing the pixel x0 (or more generally the coordinates in the coordinate system associated with the image capturing device 4) corresponding to the distal end 2b, it is easy to use the depth map to determine the measured distance d0 associated with this pixel, from the depth map shown in FIG. 3C. Next, the pixel and distance information is converted to determine the coordinates of the distal end 2b of the rod 2, which is converted to obtain the force applied to the body 3 at the measurement instant, i.e., at the instant at which the image and the depth map were acquired. If a plurality of images and depth maps have been acquired, the substeps can be repeated on each of the instances of the plurality, and the plurality of corresponding forces can be determined.

It will be noted that the information provided by the depth map, here, for each pixel of the digital image, the distance separating the exposed surface of the body 3 from the image capturing device 4, is the combination of two phenomena: the deformation of the body 3, which tends to move its surface away from the measuring device 1, and the deformation of the rod 2, which tends to bring the measuring device 1 closer to the body 3. However, the estimate of the force applied depends only on this last distance (or more precisely on the position (x, y, z) of the distal end 2b of the rod 2 with respect to the measuring device 1), so that the method can estimate this force unequivocally.

### Method for Characterizing the Rigidity of a Body

To be complete, however, it should be noted that the information provided by a single measurement (i.e., a depth map and, possibly, a digital image) is not sufficient on its own to characterize the rigidity of the body 3 being tested at the point of contact.

It is first recalled that the rigidity of a body can be characterized in multiple ways. It can be represented by, for example, the deformation distance of the body at the point of contact, according to the force applied, for example, of increasing intensities or of different orientations. The term “deformation distance” denotes the distance D between the point of contact when no force is applied and this point of contact when a determined force is applied by the measuring device 1, as made visible in FIG. 2A.

Alternatively, this deformation can be characterized and represented by a deformation profile (for example, established from the points (xi, zi) of the depth map, to take the example of FIGS. 3A-3C) of the body 3 near the point of contact, rather than by a simple deformation distance D at the point of contact.

Irrespective of how the rigidity of the body 3 is characterized, it is based on the availability of a plurality of depth maps, making it possible to evaluate the deformation of the body 3 for at least two different applied forces. Advantageously, there is a first map corresponding to the natural shape of the body 3 (in the absence of application of any force) and at least one second map corresponding to the shape of the body 3 when the measuring device 1 applies at least one determined force, according to its 3 components fx, fy, fz.

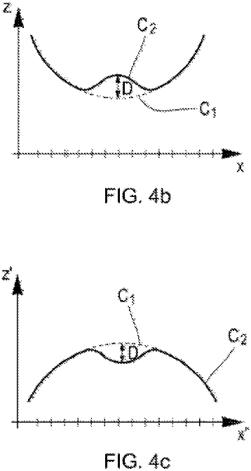

By way of example, FIG. 4A shows in dotted lines the depth map C1 produced by the measuring device 1 of FIG. 2A when the distal end 2b of the flexible rod 2 is in contact with the body 3, without, however, exerting any force on this body (zero applied force). The depth map therefore defines the undistorted profile of this body 3, in the field of view of the image capturing device 4. This depth map shows, at the point of contact between the rod 2 and the body 3, the distance d0, corresponding to the distance separating the distal end 2b of the rod 2 from the head 1b of the measuring device 1, when this rod 2 is not bent.

This same FIG. 4A shows, in solid lines, the depth map C2 produced by the measuring device when the distal end 2b of the flexible rod 2 exerts a force on the body 3, as shown in FIG. 2A. Since the rod 2 is bent, the distance d1 separating the end 2b of the rod 2 and the body 3 is reduced, less than the distance d0. Moreover, the measuring device 1 having approached the body 3 being tested overall in order to apply a deformation force thereto, the depth map C2 displays magnitudes that are generally smaller than those of the depth map C1 of the non-deformed body. The application of the previously described measuring method makes it possible to identify the force applied to the body according to its three components (fx, fy, fz).

It is observed in FIG. 2A that the body 3 is deformed near the point of contact, but that this is not the case in the peripheral zones Z of this vicinity. However, a shift is introduced between the depth maps shown in FIG. 4A.

Thus, during a characterization method according to the present disclosure, the deformation profiles of the body 3 established using the first map and the second depth map (or this first and second map directly) are readjusted with respect to each other. More generally, all the depth maps C1, C2 that have been acquired during the method are readjusted with respect to each other. In this way, the profiles produced by the image capturing device 4 carried by the head 1b of the measuring device 1 are readjusted with respect to each other.

This readjusting step can include digitally trying to make the non-deformed zones of the depth maps C1, C2 match one another, respectively, for example, at least one peripheral zone Z of FIG. 4A. An undeformed zone is sufficiently far from the point of contact that its shift from one map to another is not due to the deformation of the body 3, but only to the displacement of the measuring device 1. FIG. 4B shows the two depth maps C1, C2 of FIG. 4A, after application of this readjusting step. FIG. 4C shows the depth maps C1, C2 replaced in a coordinate system (x′, z′) linked to the body 3 being tested, to present the undeformed and deformed profile of the body 3.

To facilitate this step of readjusting the depth maps, it is possible to provide the body 3 with visual markers, referred to as readjusting markers, that can be identified in the digital images provided by the image capturing device 4. These markers can be useful, in particular, for detecting a deformation presenting a rotational component of the body 3, which would not be detectable on the depth maps.

Once the depth maps have been readjusted with respect to each other, it is possible to characterize the rigidity of the body 3, for example, by identifying the deformation distance D or the deformation profile (which appears on the depth maps of FIG. 4A and on the profile maps in FIG. 4C), this distance D or this profile being associated with the determined force f applied to the body. By way of complementary example of the characterization of the rigidity of the body 3, it is possible to provide the displacement vector of the point of contact of the rod 2 on the body 3, this vector being developed by taking the difference between the coordinates of the distal end 2b of the rod 2 in the two readjusted depth maps. The vector can be associated with the applied force determined during the measuring method to fully characterize the body 3.

Of course, the present disclosure is not limited to the embodiment described, and it is possible to add alternative embodiments without departing from the scope of the invention as defined by the claims.

In particular, the system and the measuring and characterization methods that have just been described can be used to characterize the rigidity of a body of any kind. It may be, in particular, a human body, but this particular disclosure is not limiting. It can also be any object of which it is desired, for example, to characterize the rigidity.

The device and the method can find numerous practical applications, for example, for developing a digital model of the characterized object, this model making it possible to represent or simulate the deformation of an object or of a body when the latter is subjected to external forces.

1. A measuring system for measuring a force exerted on a body; the measuring system comprising a measuring device and calculation means, the measuring device comprising:
a flexible rod having a proximal end and a distal end configured to contact the body and to apply a force tending to deform the body near a point of contact, the flexible rod configured to bend as soon as it applies a force by the distal end of the flexible rod to the body; and
an image capturing device secured to the proximal end and oriented toward the distal end of the flexible rod to provide at least one depth map of the body near the distal end of the flexible rod and the distal end; and
wherein the calculation means are configured to determine, using the depth map of the distal end of the rod, a force exerted on the body by the distal end of the flexible rod.

2. The system of claim 1, wherein the flexible rod is configured to apply a force between 0.1 N and 10 N.

3. The system of claim 2, wherein the measuring device comprises a switch to actuate the image capturing device.

4. The system of claim 3, further comprising a gripping handle secured to the image capturing device and to the flexible rod.

5. The system of claim 4, wherein the switch is in the form of a trigger.

6. The system of claim 5, wherein the calculation means comprise a transmission unit and a remote calculation device, the transmission unit being configured to exchange data with the remote calculation device.

7. The system of claim 1, wherein the calculation means comprise a calculation unit configured to process the depth map.

8. The system of claim 1, wherein the image capturing device also provides a digital image of the body near the distal end of the flexible rod.

9. The system of claim 1, wherein the distal end comprises a tip having a color identifiable in an image provided by the image capturing device.

10. The system of claim 1, wherein the distal end comprises a tip having a shape identifiable on a map provided by the image capturing device.

11. A method for measuring a force exerted on a body, the method comprising:
providing a measuring system for measuring the force exerted on the body, the measuring system comprising a measuring device and calculation means, the measuring device including:
a flexible rod having a proximal end and a distal end configured to contact the body and to apply a force tending to deform the body near a point of contact, the flexible rod configured to bend as soon as it applies a force by the distal end of the flexible rod to the body; and
an image capturing device secured to the proximal end and oriented toward the distal end of the flexible rod to provide at least one depth map of the body near the distal end of the flexible rod, and the distal end; and
wherein the calculation means are configured to determine, using the depth map of the distal end of the rod, a force exerted on the body by the distal end of the flexible rod;
contacting the body with the distal end of the rod at a point of contact and applying a force tending to deform the body near the point of contact;
acquiring, at a measurement instant, at least one depth map while applying the force tending to deform the body near the point of contact; and
processing the depth map to establish the force exerted on the body.

12. The method of claim 11, wherein the processing the depth map comprises locating the distal end of the rod in the depth map and providing coordinates of the distal end in a coordinate system associated with the measuring device.

13. The method of claim 11, further comprising calibrating the measuring device prior to acquiring the at least one depth map.

14. A method for characterizing a rigidity of a body, comprising:
performing a measuring method according to claim 11; and
acquiring, during the measuring method, a plurality of depth maps at successive measurement instants.

15. The method of claim 14, further comprising readjusting the plurality of depth maps.

16. The method of claim 16, further comprising providing the body with readjusting markers configured to allow the plurality of depth maps to be readjusted.

17. The system of claim 1, wherein the measuring device comprises a switch to actuate the image capturing device.

18. The system of claim 1, further comprising a gripping handle secured to the image capturing device and to the flexible rod.

19. The system of claim 1, wherein the measuring device comprises a trigger on a handle, the trigger configured to actuate the image capturing device.

20. The system of claim 1, wherein the calculation means comprise a transmission unit and a remote calculation device, the transmission unit being configured to exchange data with the remote calculation device.
